(19) United States
(12) Patent Application Publication
(10) Pub. No.: US 2020/0375505 A1
(43) Pub. Date: Dec. 3, 2020

(54) METHOD AND APPARATUS FOR HEALTH PREDICTION BY ANALYZING BODY BEHAVIOUR PATTERN

(71) Applicant: Next Step Dynamics AB, Malmö (SE)

(72) Inventors: Karthik SRINIVASAN, Malmö (SE); Nooria DARIAB, Malmö (SE)

(21) Appl. No.: 16/487,195

(22) PCT Filed: Feb. 21, 2018
(86) PCT No.: PCT/SE2018/050164

§ 371 Date: Aug. 20, 2019

(30) Foreign Application Priority Data
Feb. 22, 2017 (SE) 1750192-5

(51) Int. Cl. A61B 5/11; G16H 40/67; G16H 50/30; G16H 50/70; G16H 15/00; G16H 50/20; A61B 5/08; A61B 5/00

(52) U.S. Cl. CPC A61B 5/1117; G16H 40/67; G16H 50/30; G16H 50/70; G16H 15/00; A61B 2562/0219; A61B 5/1126; A61B 5/0816; A61B 5/7275; A61B 5/743; A61B 5/7475; G16H 50/20

(57) ABSTRACT

The disclosure proposes an electronic device and a method performed in the electronic device for collecting a sensor data from live at least one sensor device, obtaining a first sensor data of the sensor data representing a first primary body behaviour pattern of the person, obtaining a second sensor data of the sensor data representing a second primary body behaviour pattern of the person, the second primary body behaviour pattern is associated with the first primary body behaviour pattern of the person and determining a sensor data difference by comparing the first sensor data with the second sensor data followed by determining a health score value based on the determined sensor data difference.

### TECHNICAL FIELD

The disclosure pertains to a method and apparatus in the field of predicting the health state of a person.

### BACKGROUND

Today many persons experience negative health effects, in particular when they are getting older in life. One common negative health effect for elderly persons is getting injured by falling down. These injuries caused by falling are sometimes devastating for the elderly person. Sometimes the injuries are difficult or not possible to recover from. The injuries are at the same time something that costs a lot of money and resources in the health care system. In the country Sweden these type of injuries cost the society around 5 Billion $US every year. In Sweden around 300,000 elderly people fall at least once per year and out of them 70,000 end up in a hospital care between 8 to 12 days. 18,000 of them gets a hip fracture.

Today a person can visit a doctor or nurse that can evaluate the person's health. This is often done on a regular basis, once per year or month, or more frequent depending on the person's current health, age, etc. At such evaluations different data is measured such as pulse, blood pressure and respiration etc. The evaluation may also conclude what condition the person is in by determining the strength and balance of the person at that very moment. Simple observations of the elderly person's general health is also done by person's in the surrounding. This can be friends and family or personnel at homes for old people. Not everyone can however detect or understand changes in the health state of the elderly person, not even the elderly person him- or herself.

There are also elderly persons who have different behaviour over time both at day and night that no-one in the surroundings is aware of. As an example, an elderly person living in a home for old people may have had a bad night with hardly no sleep one night, e.g. due to new medication, but independent of that the personnel at the home for old people where the elderly person lives, wakes the elderly person up in the morning after too little sleep not knowing that the elderly person has been awake most of the night and just recently fallen to sleep.

### SUMMARY

Today there is a demand on predicting a health state of a person, relating to if a person is going to be exposed to a negative health effect. Without any indication of the persons health state the negative health effect is very difficult to avoid. Instead of waiting for the negative health effect to happen, e.g. a person getting injured by falling, there is a demand on predicting if e.g. risk of falling is increasing. This is of course of great value for the person him- or herself but also of great value for the society when it comes to money spent on health care due to persons exposed to a negative health effect such as injury's caused by falling. Another positive effect is that family members can keep an eye on the elderly person and know that he or she is doing well.

An object of the present disclosure is to provide a method and a device which seek to mitigate, alleviate, or eliminate one or more of the above-identified deficiencies in the art and disadvantages singly or in any combination.

The disclosure proposes a method performed in an electronic device comprising at least one sensor device configured to be attached to a body of a person for determining a health state of the person. The method comprising collecting a sensor data from the at least one sensor device and obtaining a first sensor data of the sensor data representing a first primary body behaviour pattern of the person. The method further comprises obtaining a second sensor data of the sensor data representing a second primary body behaviour pattern of the person, the second primary body behaviour pattern is associated with the first primary body behaviour pattern of the person. This is then followed by determining a sensor data difference by comparing the first sensor data with the second sensor data and then determining a health score value based on the determined sensor data difference. An advantage with the method is that the health score value gives an indication on a person's health and hence the risk of being exposed to a negative health effect. Changes in a certain body behaviour pattern is hence monitored and quantified in a health score value.

According to some aspects of the disclosure, the method further comprising generating a graphical representation of a health state of the person based on the health score value and displaying the graphical representation of the health state of the person via a graphical user interface on a display. The graphical representation of the person's health state can easily be understood by any person, not necessarily a Doctor, but also nursing staff and even friends or family members or the person him/herself.

According to some aspects of the disclosure, the method further comprising obtaining a first duration time for the first primary body behaviour pattern of the person and then obtaining a second duration time for the second primary body behaviour pattern of the person. This is then followed by determining a duration time difference by comparing the first duration time with the second duration time and then determining a health score value based on the determined sensor data difference and/or the determined duration time difference. An advantage with the method is that the health score value gives an indication on the current state of the person's health and hence the risk of being exposed to a negative health effect. A change in time to perform a certain body behaviour pattern is monitored and quantified in a health score value and can be an indication of the risk of being exposed to a negative health effect.

According to some aspects of the disclosure, collecting the sensor data from the at least one sensor device comprises sampling of the sensor data at a predefined sampling frequency. In this way the battery consumption of the electronic device due to the sampling can be controlled. The sampling frequency also affects the accuracy of the collected sensor data and sampling frequency can be adapted thereafter.

According to some aspects of the disclosure, collecting the sensor data from the at least one sensor device comprises sampling of the sensor data at an adapted sampling frequency, the adapted sampling frequency being dependent on the collected sensor data. Hence, the sampling frequency can be adjusted to be e.g. less frequent so that battery consumption of the electronic becomes lower when the sensor data difference is small. The sampling frequency also affects the accuracy of the collected sensor data and sampling frequency can be adapted accordingly.

According to some aspects of the disclosure, the sensor data is one or more of movement data, pulse data, force data, location data or temperature data. In this way the sensor data can be quantified with respect to changes in the person's body.

According to some aspects of the disclosure the primary body behaviour pattern is representing a certain movement characteristics of the person. Hence changes in a certain movement characteristics is monitored and quantified in a health score value, such as “getting in upright position from sitting down on a chair” or “getting out of the bed”.

According to some aspects of the disclosure, the primary body behaviour pattern is representing a certain pulse characteristics of the person. Hence changes in a certain pulse characteristics is monitored and quantified in a health score value. Thus the first primary body behaviour pattern may represent a certain pulse characteristic of the person on a first day and the second primary body behaviour pattern may represent another certain pulse characteristic of the person on another second day, such as on the following day or a week later etc. Thus by comparing the pulse characteristic for the same primary body behaviour pattern of the person, e.g. “getting out of the bed”, for the first day and for the second day, changes in a the pulse characteristics for this primary body behaviour pattern is monitored and quantified in a health score value.

According to some aspects of the disclosure, determining the health score value comprises using at least one sensor data. This means that plural sensor data can be used to calculate the health score value.

The disclosure further proposes an electronic device, comprising at least one sensor device, configured to be attached to a body of a person for determining a health state of the person. The electronic device comprises a memory and a processing circuitry that is configured to cause the electronic device to collect a sensor data from the at least one sensor device and obtain a first sensor data of the sensor data representing a first primary body behaviour pattern the person. The memory and the processing circuitry of the electronic device is further configured to cause the electronic device to obtain a second sensor data of the sensor data representing a second primary body behaviour pattern of the person, the second primary body behaviour pattern is associated with the first primary body behaviour pattern of the person and then determine a sensor data difference by comparing the first sensor data with the second sensor data and then determine a health score value based on the determined sensor data difference. An advantage with the electronic device is that the health score value gives an indication on the persons health and hence the risk of being exposed to a negative health effect. Changes in a certain body behaviour pattern is hence monitored and quantified in a health score value and can be an indication of the risk of being exposed to a negative health effect.

The present invention relates to different aspects including the method described above and in the following, and corresponding methods, electronic devices, systems, networks, uses and/or product means, each yielding one or more of the benefits and advantages described in connection with the first mentioned aspect, and each having one or more embodiments corresponding to the embodiments described in connection with the first mentioned aspect and/or disclosed in the appended claims.

### BRIEF DESCRIPTION OF THE DRAWINGS

The foregoing will be apparent from the following more particular description of the example embodiments, as illustrated in the accompanying drawings in which like reference characters refer to the same parts throughout the different views. The drawings are not necessarily to scale, emphasis instead being placed upon illustrating the example embodiments.

FIG. 1 illustrates an exemplary system suitable for implementing the proposed method.

FIG. 2 illustrates a flow chart of the method steps according to some aspects of the disclosure.

FIG. 3 illustrates graphical representation of activity based on the health score value according to some aspects of the disclosure.

FIG. 4 illustrates graphical representation of strength based on the health score value according to some aspects of the disclosure.

FIG. 5 illustrates graphical representation of balance based on the health score value according to some aspects of the disclosure.

FIGS. 6a and 6b illustrates a primary body behaviour pattern according to some aspects of the disclosure.

### DETAILED DESCRIPTION

Aspects of the present disclosure will be described more fully hereinafter with reference to the accompanying drawings. The method and device disclosed herein can, however, be realized in many different forms and should not be construed as being limited to the aspects set forth herein. Like numbers in the drawings refer to like elements throughout.

The terminology used herein is for the purpose of describing particular aspects of the disclosure only, and is not intended to limit the disclosure. As used herein, the singular forms “a”, “an” and “the” are intended to include the plural forms as well, unless the context clearly indicates otherwise.

In some implementations and according to some aspects of the disclosure, the functions or steps noted in the blocks can occur out of the order noted in the operational illustrations. For example, two blocks shown in succession can in fact be executed substantially concurrently or the blocks can sometimes be executed in the reverse order, depending upon the functionality/acts involved.

In the drawings and specification, there have been disclosed exemplary aspects of the disclosure. However, many variations and modifications can be made to these aspects without substantially departing from the principles of the present disclosure. Thus, the disclosure should be regarded as illustrative rather than restrictive, and not as being limited to the particular aspects discussed above. Accordingly, although specific terms are employed, they are used in a generic and descriptive sense only and not for purposes of limitation.

It should be noted that the word “comprising” does not necessarily exclude the presence of other elements or steps than those listed and the words “a” or “an” preceding an element do not exclude the presence of a plurality of such elements. It should further be noted that any reference signs do not limit the scope of the claims, that the example embodiments may be implemented at least in part by means of both hardware and software, and that several “means”, “units” or “devices” may be represented by the same item of hardware.

Today many persons experience negative health effects, in particular when they are getting older in life. Not everyone can detect or understand changes in the health state of an elderly person, not even the elderly person him- or herself. The inventors have identified that there is a need for a solution where one can observe current health state of a person and predict a health state of a person thanks to observations of the health state over time. This would help to understand if a person is going to be exposed to a negative health effect or not.

The inventors realized that by collecting sensor data related to a body behaviour pattern, and timestamp and store that sensor data for further comparison with new sensor data associated with the same body behaviour pattern, one can determine a difference over time. With this information one can determine a health score value. This health score value can be represented in a graphical representation A, B, C, D, E, F, G, H via a graphical user interface on a display. The visualization on the display makes it easy for anyone to understand if a person is likely to be exposed to a negative health effect.

The disclosure proposes a method performed in an electronic device 100 that both now will be described in more detail with reference to the figures.

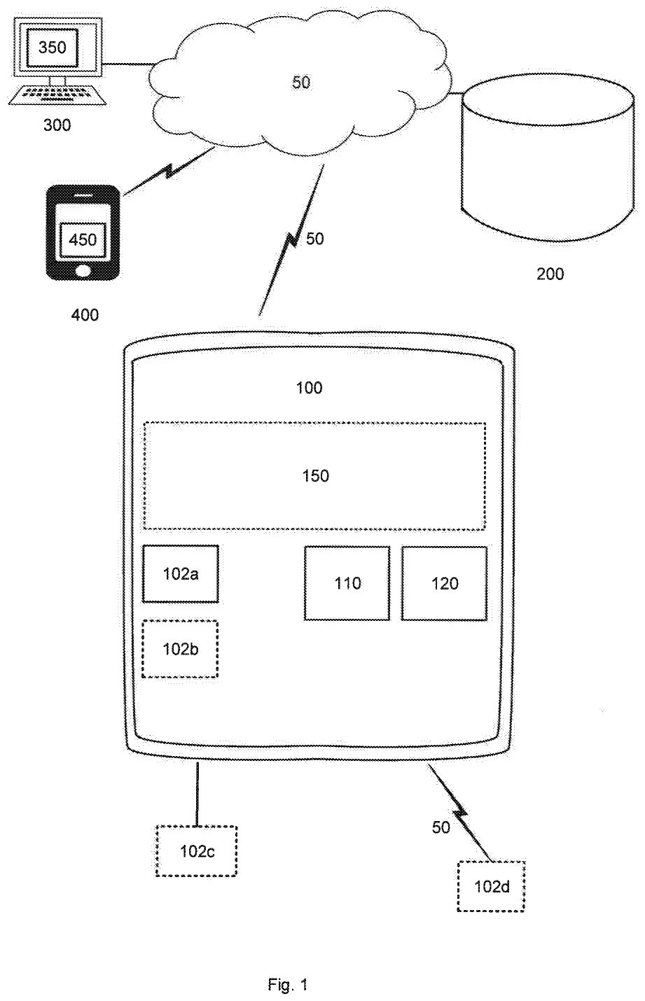

FIG. 1 illustrates an exemplary system suitable for implementing the proposed method. The system comprises the electronic device 100.

According to some embodiments of the disclosure the method is performed in an electronic device 100 comprising a memory 110 and a processing circuitry 120. According to some aspects of the disclosure the electronic device 100 further comprising a display 150 for presenting a graphical user interface. The memory 110 can be a Random-access Memory, RAM; a Flash memory; a hard disk; or any storage medium that can be electrically erased and reprogrammed. The processing circuitry 120 can be a Central Processing Unit, CPU, or any processing unit carrying out instructions of a computer program or operating system.

The electronic device 100 can be in form of a portable electronic device. The electronic device 100 can have a design and shape as any wearable device, like e.g. a watch, wristband, amulet, neckless, belt, strap or similar. According to some aspects the electronic device 100 is attached to a body of a person in order to monitor data corresponding to that person.

The electronic device 100 is in one example connected to at least another electronic device such as a server 200, personal computer 300 or a smartphone 400 via a communication network 50. The personal computer 300 or a smartphone 400 comprising at least one display 350, 450 for providing a graphical user interface. In one example the communication network 50 is a standardized wireless local area network such as a Wireless Local Area Network, WLAN, Bluetooth™, ZigBee, Ultra-Wideband, Near Field Communication, NFC, Radio Frequency Identification, RFID, or similar network. In one example the communication network 50 is a standardized wireless wide area network such as a Global System for Mobile Communications, GSM, Extended GSM, General Packet Radio Service, GPRS, Enhanced Data Rates for GSM Evolution, EDGE, Wideband Code Division Multiple Access, WCDMA, Long Term Evolution, LTE, Narrowband-IoT, 5G, Worldwide Interoperability for Microwave Access, WiMAX or Ultra Mobile Broadband, UMB or similar network. The communication network 50 can also be a combination of both a local area network and a wide area network. The communication network 50 can also be a wired network. According to some aspects of the disclosure the communication network 50 is defined by common Internet Protocols.

According to some aspects a sensor device 102a, 102b, 102c, 102d can be any of: a motion sensor such as an accelerometer or a gyroscope for detecting movements and/or relative movement, acceleration and position; a temperature sensor, for measuring the temperature; a pulse sensor for measuring the pulse, beats per minute, of a person; a respiration sensor for measuring the breathing of a person; a hygrometer, for measuring the humidity; a barometer, for measuring the air pressure; a light sensor for measuring light conditions; a camera for capturing images and video; a microphone for recording any sound such as voice; a speech recognition sensor, for identifying a person's voice; a compass, for finding a relative direction; a Global Positioning System, GPS, receiver for determining the geographical position; a pressure sensor for e.g. measuring the force on the display or on any other surface of the electronic device 100; a Body Area Network, BAN, sensor for measuring information sent via BAN; a tremor sensor for sensing a body tremor occurring in a human body; a smell sensor, for sensing different smells; a touch screen sensor for input and output of information; or any other sensor.

A sensor device 102a, 102b, 102c, 102d can also be a standalone device that is connected to the electronic device 100 either via a cable 102c or wirelessly via a wireless local area network e.g. WLAN or Bluetooth 102d. The sensor device 102a, 102b, 102c, 102d can also be integrated in other devices, e.g. in any Internet of things device such as a medical device, e.g. an electrocardiogram apparatus or a hearing aid device, via a cable 102c or wirelessly 102d. A sensor device 102a, 102b, 102c, 102d could also be any standalone device that has a sensor.

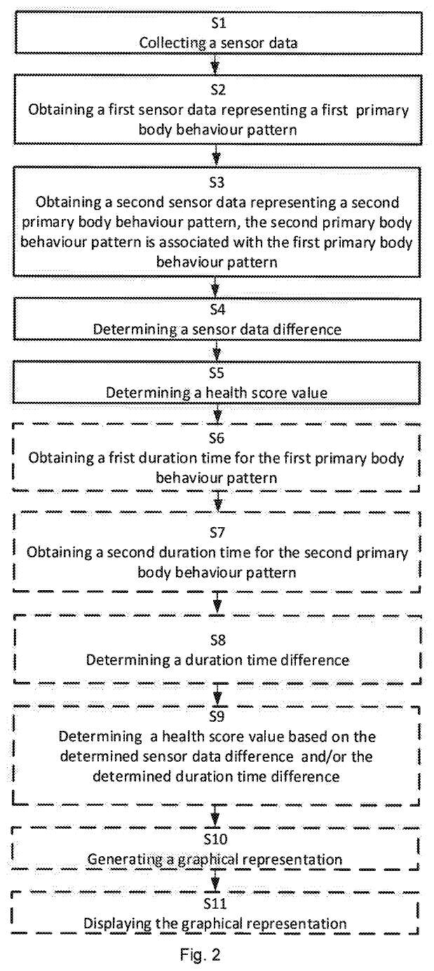

Reference is now made to FIG. 2. The disclosure proposes a method performed in an electronic device 100 comprising at least one sensor device 102a, 102b, 102c, 102d configured to be attached to a body of a person for determining a health state of the person. The method comprising collecting S1 a sensor data from the at least one sensor device 102a, 102b, 102c, 102d. According to some aspects of the disclosure plural sensor devices 102a, 102b, 102c, 102d are used for collecting different types of sensor data that together forms the sensor data. According to some aspects of the disclosure, collecting sensor data comprises collecting data from plural sensor devices 102a, 102b, 102c, 102d. According to some aspects of the disclosure the collected sensor data is timestamped and stored. The timestamped sensor data may either be stored locally in the electronic device 100 or remotely in e.g. a server 200 or personal computer 300.

According to some aspects the sensor data is any or plural of: movement data; pulse data; temperature data; force data, strength data and/or respiration data. The method further comprises obtaining S2 a first sensor data sd1 of the sensor data representing a first primary body behaviour pattern 1BBP1 of the person. According to some aspects, a body behaviour pattern is representing a certain movement characteristics of the person. According to some aspects, a body behaviour pattern is representing a certain pulse characteristics of the person. In one example, non-movement is also a kind of movement characteristics. In particular, a person that is not moving when lying down may be exposed to the negative health effect of bedsore. According to some aspects of the invention a body behaviour pattern is representing a combination of different characteristics of a person. In one example a body behaviour pattern is representing a certain movement and a certain pulse characteristics of the person. According to some aspects of the disclosure, determining the health score value comprises using at least one sensor data. This means that plural sensor data can be used to calculate the health score value.

In one example, a certain body behaviour pattern is the movement characteristics described by sensors when getting out of bed form lying down to standing up in an upright position.

According to some aspects this particular movement is defined by plural sensor devices 102a, 102b, 102c, 102d. One sensor device 102a, 102b, 102c, 102d measures e.g. the relative movement of the person by use of an accelerometer or a gyroscope. Another sensor device 102a, 102b, 102c, 102d measures the change in altitude by use of a barometer, measuring the air pressure. A further sensor device 102a, 102b, 102c, 102d measures the pulse of the person, by use of a pulse sensor. According to some aspects of the disclosure a certain body behaviour pattern can, within a certain confidence interval, be described by a function f(x,y,z) depending on the sensor data collected from plural sensor devices 102a, 102b, 102c, 102d. In the example when the body behaviour pattern is getting out of bed form lying down to standing up in an upright position, the function can be described by f(x,y,z) where:

x=accelerometer data

y=altitude data

z=pulse data

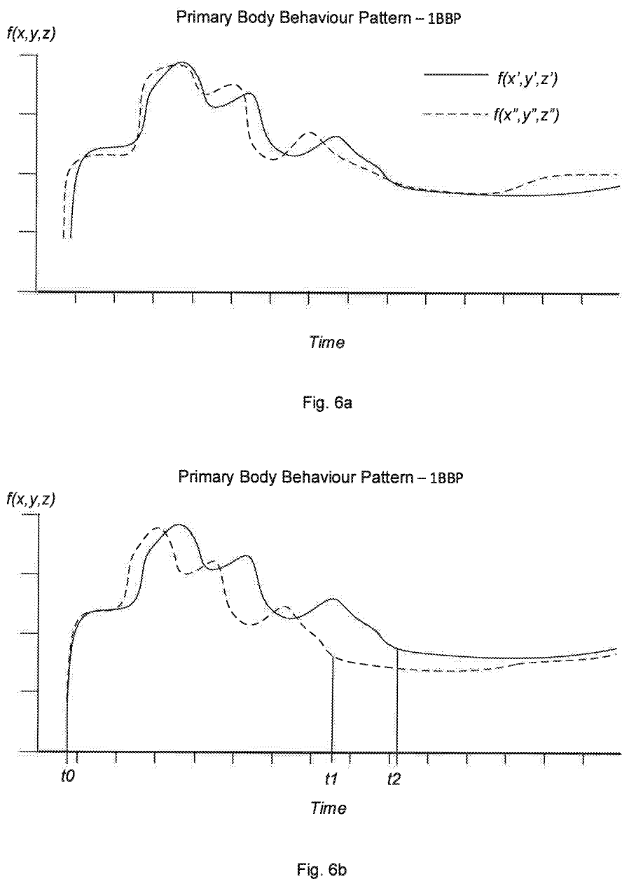

According to some aspects of the disclosure the method further comprises obtaining a first sensor data sd1 of the collected sensor data and using the first sensor data sd1 for calculating a function. The outcome of the calculation is used to define a first primary body behaviour pattern. In one example the function can e.g. be f(x,y,z) and the outcome can be a curve that is representing a first primary body behaviour pattern 1BBP1 of the person. An example of such curve is illustrated in FIG. 6a.

According to some aspects there are plural different body behaviour patterns such as primary, secondary, tertiary, quaternary, quinary, senary, septenary octonary, nonary and denary body behaviour patterns etc. Some examples:

Primary body behaviour pattern, 1BBP—getting out of bed form lying down to standing up in an upright position;

Secondary body behaviour pattern, 2BBP—getting up from sitting on a chair to an upright standing position;

Tertiary body behaviour pattern, 3BBP—sitting down on a chair from an upright standing position;

Quaternary body behaviour pattern, 4BBP—walking;

Quinary body behaviour pattern, 5BBP—walking with a stick;

Senary body behaviour pattern, 6BBP—walking with a walking frame;

Septenary body behaviour pattern, 7BBP—walking downwards in stairs;

Octonary body behaviour pattern, 8BBP—walking upwards in stairs;

Nonary body behaviour pattern, 9BBP—sleeping; etc.

According to some aspects of the disclosure all the collected sensor data is sent to a server 200. The server may be connected to plural electronic devices 100 over the communication network 50 and collect sensor data from the plural electronic devices 100. According to some aspects, a certain body behaviour pattern can be defined by sensor data that is collected and aggregated from plural persons.

According to some aspects of the disclosure a certain body behaviour pattern can be defined by sensor data that has only been collected with respect to a certain person. In one example each body behaviour pattern have to be defined manually by a user inputting data to the electronic device 100. In one example the electronic device 100 can be self-trained to label certain body behaviour patterns. In one example the electronic device 100 itself learns certain body behaviour patterns and differentiate them from each other without knowing exactly what the body behaviour pattern is actually representing in real life. In one example labeling or naming of certain body behaviour patterns is done manually either by entering data into the electronic device 100 by input means on the electronic device 100 or by inputting data by an operator of e.g. a personal computer 300 or portable device 400 that is in connection with the electronic device 100 over the communication network 50. In one example the labeling or naming of certain body behaviour patterns is done automatically by retrieving a name or label from a server 200.

According to some aspects the electronic device 100 collects sensor data from the at least one sensor device 102a, 102b, 102c, 102d and stores the all the sensor data in the memory 110. According to some aspects the electronic device 100 collects sensor data from the at least one sensor device 102a, 102b, 102c, 102d and stores the all the sensor data in a server 200 connected to the electronic device 100 over a communication network 50.

Body behaviour pattern may be defined by various of sensor data. The sensor data can for example be related to pulse, breathing, spasm, walking, sleeping. E.g. a non-movement when sleeping meaning no change of movement sensor data can also be sensor data that is of relevance this time of the day since even if the person is sleeping, a certain body behaviour pattern may be expected when comparing with previous nights.

The method further comprises obtaining S3 a second sensor data sd2 of the sensor data representing a second primary body behaviour pattern 1BBP2 of the person, the second primary body behaviour pattern 1BBP2 is associated with the first primary body behaviour pattern of the person 1BBP1 of the person.

As previously mentioned, a certain body behaviour pattern can be described by a function f(x,y,z) depending on obtained sensor data. In order to recognize that the second sensor data sd2 is representing a body behaviour pattern that is associated with the primary body behaviour pattern, the second sensor data sd2 is used as input when calculating the function f(x,y,z).

In one example the function f(x′,y′,z′) use a first sensor data sd1 and outputs a curve describing a first certain body behaviour pattern. In one example the function f(x″,y″,z″) use a second sensor data sd2 and outputs a curve describing a second certain body behaviour pattern. According to some aspects of the disclosure the function f(x′,y′,z′) is defined as representing the primary body behaviour pattern 1BBP i.e. “getting out of bed form lying down to standing up in an upright position”.

The output from the calculations of f(x′,y′,z′) and f(x″,y″,z″) are compared and if both outputs, e.g. both curves, fall within a certain confidence interval, e.g. 85%, then the second certain body behaviour pattern is identified as a primary body behaviour pattern, and in this case then defined as the second primary body behaviour pattern 1BBP2. The second primary body behaviour pattern 1BBP2 is hence associated with the first primary body behaviour pattern 1BBP1. An example of when both outputs, e.g. curves, fall within a certain confidence interval is visualized in FIG. 6a and FIG. 6b that shows two example curves that fall within a certain confidence interval of the primary body behaviour pattern.

According to some aspects of the disclosure the method further comprising continuously obtaining and comparing sensor data in order to identify a certain body behaviour pattern. According to some aspects of the disclosure the method further comprising continuously obtaining and comparing existing collected sensor data with new collected sensor data in order to identify sensor data that is representing a primary body behaviour pattern.

In one example the second primary body behaviour pattern 1BBP2 is detected by at least one sensor device 102a, 102b, 102c, 102d in time after the first primary body behaviour pattern 1BBP1 is detected. The second primary body behaviour pattern 1BBP2 can in some aspects occur at approximately the same time of the day as the first primary body behaviour pattern 1BBP1. In one example when the primary body behaviour pattern 1BBP is “getting out of bed form lying down to standing up in an upright position” it can occur more frequent. Dependent on the body behaviour pattern it can occur less or more frequent or less or more repeatedly at approximately the same time of the day. According to some aspects, the time of the day does matter for comparison with first primary body behaviour pattern and the second primary body behaviour pattern. E.g. a person may behave differently if he or she is getting out of the bed in the morning or if he or she is getting out of the bed after a nap in the afternoon.

The method is then followed by determining S4 a sensor data difference by comparing the first sensor data sd1 with the second sensor data sd2. In the previous mentioned example the primary body behaviour pattern 1BBP is “getting out of bed form lying down to standing up in an upright position”. In one example the first primary body behaviour pattern 1BBP1 is sensor data obtained when getting out of bed on Wednesday morning at 08:05 and the second primary body behaviour pattern 1BBP2 is sensor data obtained when getting out bed on Thursday morning at 07:40. In this example, illustrated in FIG. 6a, the function f(x′,y′,z′) of the first primary body behaviour pattern 1BBP1 is describing, within a certain confidence interval, a similar curve as the function f(x″,y″,z″) of the second primary body behaviour pattern 1BBP2. The sensor data difference is determined by comparing the outcome of the two functions, and in particular quantified by the different values from the first sensor data sd1 and the second sensor data sd2.

The method then determining S5 a health score value based on the determined sensor data difference. According to some aspects of the disclosure the health score value is further dependent on previously collected sensor data. According to some aspects of the disclosure the health score value is further dependent on at least one or plural of the factors time of day, gender, age or medicine.

According to some aspects of the disclosure the health score value is determined by calculating a function and using the outcome of the calculation to define the health score value. In one example the function can e.g. be f(sd1,sd2,td1,td2,b,c,d) and the outcome can be a value that is representing the health score value. The parameters can be a first sensor data, sd1; a second sensor data, sd2; first duration time, td1; a second duration time, td2; a time of day parameter, b; an average value, c; a medicine factor, d. It is understood that plural mathematical functions can be utilized and using different parameters. According to some aspects of the disclosure the health score value is further dependent on at least one or plural of the parameters time of day, gender, age or medicine.

An advantage with the method is that the health score value gives an indication on a person's health and hence the risk of being exposed to a negative health effect. In one aspect changes in a certain body behaviour pattern is monitored and quantified in a health score value.

According to some aspects of the disclosure, as illustrated in FIG. 2, the method further comprising obtaining S6 a first duration time for the first primary body behaviour pattern 1BBP1 of the person and then obtaining S7 a second duration time for the second primary body behaviour pattern 1BBP2 of the person. In the previous mentioned example the primary body behaviour pattern 1BBP is “getting out of bed form lying down to standing up in an upright position”. As illustrated in FIG. 6b, the time t1 for this particular body behaviour pattern is at one occasion t1 and at another occasion t2. In one example, a person may one morning be showing less strength, e.g. obtained by collected sensor data from an accelerometer, and have a different pulse compare to normal, which may result in that it also takes longer time to get out of the bed.

The method is then followed by determining S8 a duration time difference by comparing the first duration time td1 with the second duration time td2 and then determining S9 a health score value based on the determined sensor data difference and/or the determined duration time difference.

This means that the health score value can further be based on a duration time difference in addition to the determined sensor data difference, as in the example when getting out of the bed with less strength and different pulse. The health score value can also only be based on the determined time difference. According to some aspects of the disclosure, the certain body behaviour pattern is individual and therefore the determined sensor data difference and/or the determined duration time difference with respect to the person using the electronic device 100 is only of interest since it is reflecting the characteristics of that person i.e. only comparing previous characteristics for the same person.

An advantage with the method is that the health score value gives an indication on the current change of a person's health and hence the risk of being exposed to a negative health effect. According to some aspects a change in time to perform a certain body behaviour pattern is monitored and quantified in a health score value.

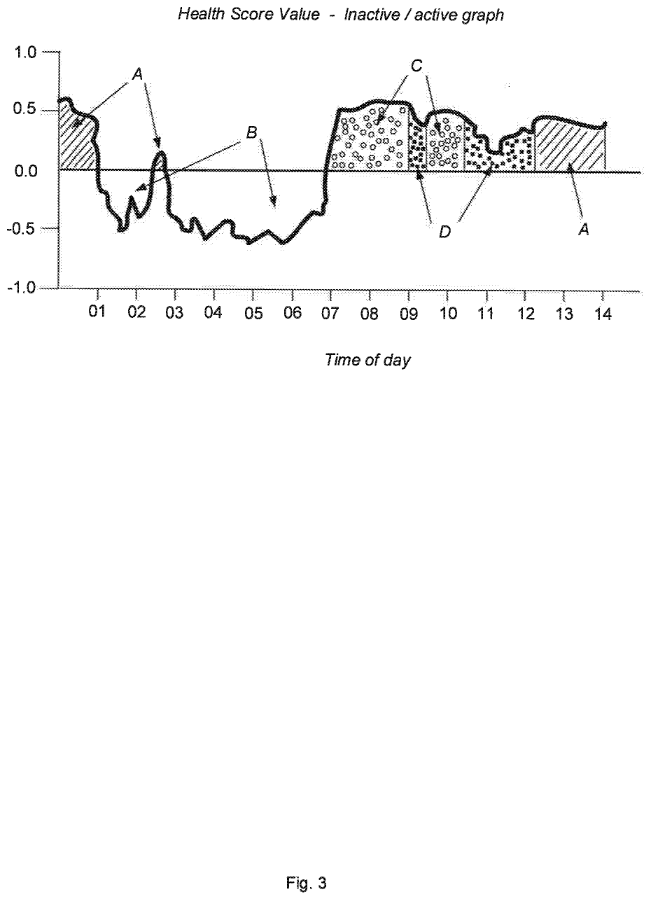

According to some aspects of the disclosure, the method further comprising generating S10 a graphical representation A, B, C, D, E, F, G, H of a health state of the person based on the health score value and displaying S11 the graphical representation A, B, C, D, E, F, G, H of the health state of the person via a graphical user interface on a display 150, 350, 450. The graphical representation A, B, C, D, E, F, G, H of the person's health state can easily be understood by any person, not necessarily a Doctor, but also nursing staff and even friends or family members. FIGS. 3-5 illustrates examples of how a graphical representation A, B, C, D, E, F, G, H of a health state of a person based on the health score value can look like. The graphical representation A, B, C, D, E, F, G, H of the person's health state can easily be understood by any person, not necessarily a Doctor, but also nursing staff and even friends or family members.

According to some aspects of the disclosure the health score value is used for initiating an action. The action can e.g. be initiating an alarm, sending a message, sending a warning flag to a system, sending a warning message to a predefined receiver or change the change graphical representation A, B, C, D, E, F, G, H.

FIG. 3 illustrates an example of an inactive/active score for a person over time of day from 01:00 in the morning to 14:00 in the afternoon. The graph has different colours that gives an observer of the graphical representation an understanding of a positive or negative health effect at a certain time of day. In FIG. 3 the area indicated as A represents a negative progress when compared to the previous day. The area B indicates normal inactivity or sleep. The area C indicates continuous walking, based on e.g. the “septenary body behaviour pattern”, 7BBP walking. The area D indicates another active movement other than walking.

In the example the graphical representation of the health state indicates that the person had a negative score in activity just before getting into bed after 01:00, maybe the person have been active too late and getting into bed too late compare to what is normal which has been considered as a negative progress. Also the person has been awake just before 03:00 at night which is considered negative compare to getting a good night sleep which may be normal. During the day the person was walking and active and experienced a progress in health effect.

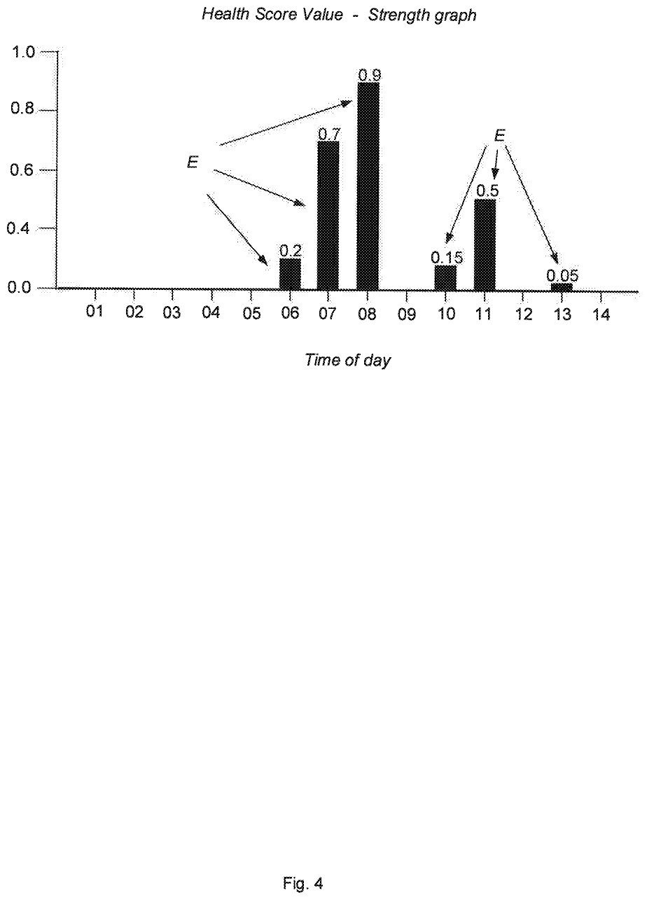

The illustration in FIG. 4 the graphical representation of the health state indicates a progress in strength over time of day compared to previous day. During hours represented with bars, 06:00-08:00 and 10:00-11:00 and at 13:00 the person had an progress in strength compare to previous day. The other hours there is no progress in strength compare to the previous day.

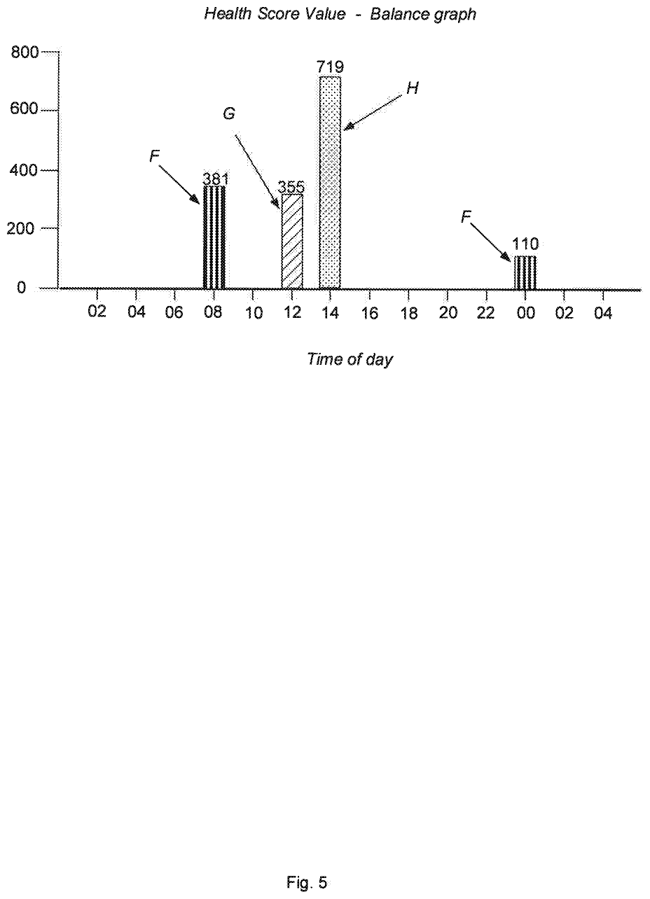

The illustration in FIG. 5 the graphical representation of the health state indicates with the area F that the person has experienced similar progress in balance when compared to the previous day. The area G indicates a decline in progress in balance when compared to the previous day. The area H indicates a better progress in balance when compared to the previous day.

According to some aspects of the disclosure, collecting the sensor data from the at least one sensor device 102a, 102b, 102c, 102d comprises sampling of the sensor data at a predefined sampling frequency. In this way the battery consumption of the electronic device 100 due to the sampling can be controlled. The electronic device 100 comprising a processing circuitry 120 and dependent on the amount of data to process, the power consumption of the electronic device 100 is affected. The more processing, the more power consumption.

According to some aspects of the disclosure, collecting the sensor data from the at least one sensor device 102a, 102b, 102c, 102d comprises sampling of the sensor data at an adapted sampling frequency, the adapted sampling frequency being dependent on the collected sensor data. Hence, the sampling frequency can be adjusted to be e.g. less frequent so that battery consumption of the electronic becomes lower when for example the sensor data difference is small. In one example there may be very little changes in the sensor data collected during night when a person is sleeping compare to during day time when the person is active and moving around. According to some aspects of the disclosure the adapted sampling frequency is dependent on the body behaviour pattern. For example if the body behaviour pattern is the Quaternary body behaviour pattern, 4BBP—“walking” the sampling can be adjusted to a certain frequency that may be higher compared to if the body behaviour pattern is the Nonary body behaviour pattern, 9BBP—“sleeping”. If the collected sensor data indicates a great variation, then sampling may be sampled at a higher frequency. If the collected sensor data is more or less the same, the sample may be sampled at a lower frequency. The sampling frequency also affects the accuracy of the collected sensor data and sampling frequency can be adapted accordingly.

According to some aspects of the disclosure, the sensor data is one or more of movement data, pulse data, force data, location data or temperature data. In this way the sensor data can be quantified with respect to changes in the physical surroundings of the person's body.

The disclosure further proposes an electronic device 100, comprising at least one sensor device 102a, 102b, 102c, 102d, configured to be attached to a body of a person for determining a health state of a person, comprising a memory 110 and a processing circuitry 120 that is configured to cause the electronic device 100 to collect a sensor data from the at least one sensor device 102a, 102b, 102c, 102d and obtain a first sensor data sd1 of the sensor data representing a first primary body behaviour pattern the person. The memory and the processing circuitry 120 of the electronic device 100 is further configured to cause the electronic device 100 to obtain a second sensor data sd2 of the sensor data representing a second primary body behaviour pattern of the person, the second primary body behaviour pattern is associated with the first primary body behaviour pattern of the person and then determine a sensor data difference by comparing the first sensor data sd1 with the second sensor data sd2 and then determine a health score value based on the determined sensor data difference. An advantage with the electronic device 100 is that the health score value gives an indication on the persons health and hence the risk of being exposed to a negative health effect. Changes in a certain body behaviour pattern is hence monitored and quantified in a health score value.

The electronic device 100 is configured to perform any of the aspects of the method described above. According to some embodiments of the disclosure, the method is carried out by instructions in a software program that is downloaded and run on the electronic device 100. In one example the software is a so called app. The app is either free or can be bought by the user of the smartphone. The same app can generate the graphical representation A, B, C, D, E, F, G, H and displaying the graphical representation A, B, C, D, E, F, G, H of the health state of the person via the graphical user interface on a display 150, 350, 450.

In the drawings and specification, there have been disclosed exemplary embodiments. However, many variations and modifications can be made to these embodiments. Accordingly, although specific terms are employed, they are used in a generic and descriptive sense only and not for purposes of limitation, the scope of the embodiments being defined by the following claims.

1. A method performed in an electronic device comprising a respiration sensor device and a motion sensor device configured to be attached to a body of a person for determining a health state of the person, the method comprising:
collecting a sensor data from the respiration sensor device and the motion sensor device;
obtaining a first sensor data of the sensor data representing a first primary body behaviour pattern of the person;
obtaining a second sensor data of the sensor data representing a second primary body behaviour pattern of the person, the second primary body behaviour pattern is associated with the first primary body behaviour pattern of the person;
determining a sensor data difference by comparing the first sensor data with the second sensor data; and
determining a health score value based on the determined sensor data difference,
wherein the sensor data is related to measuring the breathing of the person and movement of the person, and the primary body behaviour pattern is one of plural different body behaviour patterns, and each body behaviour pattern is described by a function that is depending on the sensor data, and the primary body behaviour pattern is representing a certain movement characteristics of the person.

2. The method according to claim 1 further comprising:
generating a graphical representation of a health state of the person based on the health score value; and
displaying the graphical representation of the health state of the person via a graphical user interface on a display.

3. The method according to claim 1, further comprising:
obtaining a first duration time for the first primary body behaviour pattern of the person;
obtaining a second duration time for the second primary body behaviour pattern of the person;
determining a duration time difference by comparing the first duration time with the second duration time; and
determining a health score value based on the determined sensor data difference and/or the determined duration time difference.

4. The method according to claim 1, wherein collecting the sensor data from the respiration sensor device and the motion sensor device comprises sampling of the sensor data at a predefined sampling frequency.

5. The method according to claim 1, wherein collecting the sensor data from the respiration sensor device and the motion sensor device comprises sampling of the sensor data at an adapted sampling frequency, the adapted sampling frequency being dependent on the collected sensor data.

6-8. (canceled)

9. The method according to claim 1, wherein determining the health score value comprises using at least one sensor data.

10. An electronic device comprising a respiration sensor device and a motion sensor device configured to be attached to a body of a person for determining a health state of the person, the electronic device comprising:
a memory;
a processing circuitry, configured to cause the electronic device to:
collect a sensor data from the respiration sensor device and the motion sensor device;
obtain a first sensor data of the sensor data representing a first primary body behaviour pattern of the person;
obtain a second sensor data of the sensor data representing a second primary body behaviour pattern of the person,
the second primary body behaviour pattern is associated with the first primary body behaviour pattern of the person;
determine a sensor data difference by comparing the first sensor data with the second sensor data; and
determine a health score value based on the determined sensor data difference,
wherein the sensor data is related to measuring the breathing of the person and movement of the person, and the primary body behaviour pattern is one of plural different body behaviour patterns, and each body behaviour pattern is described by a function that is depending on the sensor data, and the primary body behaviour pattern is representing a certain movement characteristics of the person.
